(19) United States
(12) Patent Application Publication
(10) Pub. No.: US 2023/0340416 A1
(43) Pub. Date: Oct. 26, 2023

(54) AUGMENTATION OF FIBROBLAST THERAPEUTIC ACTIVITY BY COMPLEMENT BLOCKADE AND/OR INHIBITION

(71) Applicant: FIGENE, LLC, Houston, TX (US)

(72) Inventor: Thomas ICHIM, San Diego, CA (US)

(21) Appl. No.: 18/005,065

(22) PCT Filed: Jul. 13, 2021
(86) PCT No.: PCT/US2021/070871

§ 371 Date: Jan. 11, 2023

Related U.S. Application Data
(60) Provisional application No. 63/051,845, filed on Jul. 14, 2020.

(51) Int. Cl. C12N 5/077; C07K 16/18

(52) U.S. Cl. CPC C12N 5/0656; C07K 16/18; C12N 2502/02; C12N 2501/999; C12N 2500/02; C12N 2500/60; C12N 2501/10; C12N 2501/20

(57) ABSTRACT

Increasing therapeutic activity of fibroblasts through suppression of complement activation is disclosed. Embodiments of the disclosure teach that viability of fibroblasts in blood and/or in vivo is increased by inhibition of complement activation. In another embodiment, the blockade of complement is utilized to enhance ability of fibroblasts to suppress inflammation, stimulate generation of T regulatory cell, and inhibit pathologic T cell responses. Other enhancements of fibroblast activity disclosed as a results of complement activation include stimulation of cytokine production, release of antimicrobial and/or antiviral proteins, as well as enhancement of regenerative activities.

### TECHNICAL FIELD

Embodiments of the disclosure encompass at least the fields of cell biology, molecular biology, and medicine.

### BACKGROUND

The blood-borne protein family of “Complement” was first discovered in the 1890s when it was found to aid or “complement” the killing of bacteria by heat-stable antibodies present in normal serum [1, 2]. The complement system consists of more than 30 proteins that are either present as soluble proteins in the blood or are present as membrane-associated proteins [3]. Activation of complement leads to a sequential cascade of enzymatic reactions, known as complement activation pathways, resulting in the formation of the potent anaphylatoxins C3a and C5a that elicit a plethora of physiological responses that range from chemoattraction to apoptosis. Initially, complement was thought to play a major role in innate immunity where a robust and rapid response is mounted against invading pathogens [4].

Recently it is becoming increasingly evident that complement also plays an important role in adaptive immunity involving T and B cells that help in elimination of pathogens [5]. One of the early studies demonstrating involvement of complement in adaptive immunity showed that the fifth component of the complement cascade, C5a, is capable of potentiating antigen- and alloantigen-induced T cell proliferative responses. It was found that the carboxyterminal arginine of C5a is not essential in order for C5a to enhance immune responses. C5a des Arg was found to augment the immune response to the level of C5a-mediated enhancement. The serum carboxypeptidase inhibitor, 2-mercaptomethyl-5-quanodinopentanoic acid, which prevents cleavage of the terminal arginine, allowed for assessment of the effects of C5a on in vitro immune responses in the presence of serum. It was shown that helper T cells are involved in C5a-mediated immuno-potentiation. Substitution of T cells by soluble T cell-replacing factors, (Fc)TRF, rendered lymphocyte cultures refractory to the enhancing properties of C5a [6].

In another study, flow cytometry analysis was used identify the complement 5a receptor (C5aR) on T cells. It was found that this is expressed at a low basal level on unstimulated T cells and was strikingly up-regulated upon PHA stimulation in a time- and dose-dependent manner. CD3+ sorted T cells as well as Jurkat T cells were shown to express C5aR mRNA as assessed by RT-PCR. In order for the scientists to demonstrate that C5a was biologically active on T cells, we investigated the chemotactic activity of C5a and observed that purified CD3+ T cells are chemotactic to C5a at nanomolar concentrations. Finally, using a combination of in situ hybridization and immunohistochemistry, the investigators showed that the T cells infiltrating the central nervous system during experimental allergic encephalomyelitis express the C5aR mRNA. These data suggest that innate inflammation may trigger T cell chemotaxis to areas of immunological need [7]. Complement components other than C5 are also involved in T cell activation. For example, in one study, allospecific immunoglobulin (Ig)G response was markedly impaired in C3− and C4−, but not in C5-deficient mice. This defect was most pronounced for second set responses. C3-deficient mice also demonstrated a decreased range of IgG isotypes. In contrast, there was no impairment of the allospecific IgM response. In functional T cell assays, the proliferative response and interferon-gamma secretion of recipient lymphocytes restimulated in vitro with donor antigen was decreased two- to threefold in C3-deficient mice [8].

The role of complement in host T cell mediated defenses also appears relevant. Indeed patients with complement genetic deficiencies are known to possess weaker T cell responses. In animals, a strong basic research study examined the CD8(+) T cell response in influenza type A virus-infected mice treated with a peptide antagonist to C5aR to test the potential role of complement components in CD8(+) T cell responses. It was demonstrated both the frequency and absolute numbers of flu-specific CD8(+) T cells are greatly reduced in C5aR antagonist-treated mice compared with untreated mice. This reduction in flu-specific CD8(+) T cells is accompanied by attenuated antiviral cytolytic activity in the lungs. These results demonstrate that the binding of the C5a component of complement to the C5a receptor plays an important role in CD8(+) T cell responses [9]. While the previous study demonstrated reduction in complement can compromise T cell immunity, another study demonstrated enhancement of complement augmented T cell responses. The investigators used mice deficient for decay accelerating factor (DAF), which breaks down complement. Compared with wild-type mice, DAF knockout (Daf-1(−/−)) mice had markedly increased expansion in the spleen of total and viral Ag-specific CD8+ T cells after acute or chronic LCMV infection. Splenocytes from LCMV-infected Daf-1(−/−) mice also displayed significantly higher killing activity than cells from wild-type mice toward viral Ag-loaded target cells, and Daf-1(−/−) mice cleared LCMV more efficiently. Importantly, deletion of the complement protein C3 or the receptor for the anaphylatoxin C5a (C5aR) from Daf-1(−/−) mice reversed the enhanced CD8+ T cell immunity phenotype. These results demonstrate that DAF is an important regulator of CD8+ T cell immunity in viral infection and that it fulfills this role by acting as a complement inhibitor to prevent virus-triggered complement activation and C5aR signaling [10]. Others studies have confirmed a role for various complement components in manipulation of T cell immunity [11-34].

The interaction between the innate and adaptive branches of the immune system have been previously described at several levels. For example, T cell activation of dendritic cells usually requires dendritic cells to mature in order to allow for proper antigen presentation and formation of the immunological synapse [35]. It is established that immature dendritic cells are generally tolerogenic, and induce T regulatory cells as opposed to proper T cell activation [36-82]. The process of immature dendritic cells stimulating suppressor T cells is well known in cancer, in which tumors inhibit dendritic cell maturation through production of factors such as VEGF, PGE-2, IL-10 and TGF-beta [83-86]. In the natural context, apoptotic cells possess phosphotidylserine on their surface, which maintains dendritic cells in immature states [87-98]. In contrast, during tissue damage, or infection, dendritic cells mature due to activation of receptors such as toll like receptors. Mature dendritic cells subsequent activate T cell immunity due to expression of both Signal 1 (MHC/antigen) and Signal 2 (costimulatory signals) [99]. Interestingly, some studies have shown that apoptotic bodies actually inhibit expression and/or signaling of toll like receptors [100-103].

At a basic level, complement activation is known to occur through three different pathways: alternate, classical, and lectin, involving proteins that mostly exist as inactive zymogens that are then sequentially cleaved and activated. All pathways of complement activation lead to cleavage of the C5 molecule generating the anaphylatoxin C5a and, C5b that subsequently forms the terminal complement complex (C5b-9). C5a exerts a predominant proinflammatory activity through interactions with the classical G-protein coupled receptor C5aR (CD88) as well as with the non-G protein coupled receptor C5L2 (GPR77), expressed on various immune and non-immune cells. C5b-9 causes cytolysis through the formation of the membrane attack complex (MAC), and sub-lytic MAC and soluble C5b-9 also possess a multitude of non-cytolytic immune functions. These two complement effectors, C5a and C5b-9, generated from C5 cleavage, are key components of the complement system responsible for propagating and/or initiating pathology in different diseases, including paroxysmal nocturnal hemoglobinuria, rheumatoid arthritis, ischemia-reperfusion injuries and neurodegenerative diseases.

The complement system is described in detail in U.S. Pat. No. 6,355,245, which is incorporated herein by reference. The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum [104, 105]. Complement components achieve their immune defensive functions by interacting in a series of intricate but precise enzymatic cleavage and membrane binding events. The resulting complement cascade leads to the production of products with opsonic, immunoregulatory, and lytic functions. The complement cascade progresses via the classical pathway or the alternative pathway. These pathways share many components and, while they differ in their initial steps, they converge and share the same “terminal complement” components (C5 through C9) responsible for the activation and destruction of target cells. The classical complement pathway is typically initiated by antibody recognition of and binding to an antigenic site on a target cell. The alternative pathway is usually antibody independent and can be initiated by certain molecules on pathogen surfaces. Both pathways converge at the point where complement component C3 is cleaved by an active protease (which is different in each pathway) to yield C3a and C3b. Other pathways activating complement attack can act later in the sequence of events leading to various aspects of complement function [106]. C3a is an anaphylatoxin. C3b binds to bacterial and other cells, as well as to certain viruses and immune complexes, and tags them for removal from the circulation. C3b in this role is known as opsonin. The opsonic function of C3b is considered to be the most important anti-infective action of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone. C3b also forms a complex with other components unique to each pathway to form classical or alternative C5 convertase, which cleaves C5 into C5a and C5b. C3 is thus regarded as the central protein in the complement reaction sequence since it is essential to both the alternative and classical pathways. This property of C3b is regulated by the serum protease Factor I, which acts on C3b to produce iC3b. While still functional as opsonin, iC3b cannot form an active C5 convertase.

C5 is a 190 kDa beta globulin found in normal serum at approximately 75 .mu.g/mL (0.4.mu.M.) C5 is glycosylated, with about 1.5-3 percent of its mass attributed to carbohydrate. Mature C5 is a heterodimer of a 999 amino acid 115 kDa alpha chain that is disulfide linked to a 656 amino acid 75 kDa beta chain. C5 is synthesized as a single chain precursor protein product of a single copy gene (Haviland et al., 1991). The cDNA sequence of the transcript of this gene predicts a secreted pro-05 precursor of 1659 amino acids along with an 18 amino acid leader sequence. The pro-05 precursor is cleaved after amino acid 655 and 659, to yield the beta chain as an amino terminal fragment (amino acid residues +1 to 655) and the alpha chain as a carboxyl terminal fragment (amino acid residues 660 to 1658), with four amino acids deleted between the two. C5a is cleaved from the alpha chain of C5 by either alternative or classical C5 convertase as an amino terminal fragment comprising the first 74 amino acids of the alpha chain (i.e., amino acid residues 660-733). Approximately 20 percent of the 11 kDa mass of C5a is attributed to carbohydrate. The cleavage site for convertase action is at or immediately adjacent to amino acid residue 733. A compound that would bind at or adjacent to this cleavage site would have the potential to block access of the C5 convertase enzymes to the cleavage site and thereby act as a complement inhibitor. C5 can also be activated by means other than C5 convertase activity. Limited trypsin digestion and acid treatment can also cleave C5 and produce active C5b. C5a is another anaphylatoxin. C5b combines with C6, C7, and C8 to form the C5b-8 complex at the surface of the target cell. Upon binding of several C9 molecules, the membrane attack complex (MAC, C5b-9, terminal complement complex-TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis. As mentioned above, C3a and C5a are anaphylatoxins. These activated complement components can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract proinflammatory granulocytes to the site of complement activation.

Although complement has been implicated in numerous states of immunity, no one has examined the effects of complement manipulation as a means of altering efficacy of fibroblast cell therapy.

### BRIEF SUMMARY

The present disclosure is directed to compositions and methods for increasing fibroblast therapeutic activity. In some embodiments, a therapeutically effective amount of fibroblasts and/or fibroblast-derived products are administered to an individual in order to provide therapeutic activity to the individual. Embodiments herein concern increasing that therapeutic activity via methods comprising the modulation of complement activation in the individual. In some embodiments, the individual is assessed for the potential for complement activation. Assessment for potential for complement activation may be performed by assaying levels of complement components in the blood, as well as determining density of molecules capable of activating complement on surface of fibroblasts, in at least some cases.

The fibroblasts may be from any source, including tissue selected from the group consisting of cord blood, Wharton's Jelly, placenta, bone marrow, adipose tissue, skin, deciduous teeth, nails, peripheral blood, omentum, and a combination thereof. The fibroblasts may be allogeneic, autologous, or xenogeneic to the individual. The fibroblasts may be plastic adherent. Adherent cells are cells that stick to plastics, such as to a tissue culture plate. The fibroblast-derived products may comprise exosomes derived from fibroblasts.

The fibroblasts may express one or more markers including markers selected from the group consisting of CD73, CD105, stage-specific embryonic antigens (SSEA) 3, SSEA 4, Tra-1-60, Tra-1-81, Oct-3/4, Cripto, gastrin-releasing peptide (GRP) receptor, podocalyxin-like protein (PODXL), Rex-1, GCTM-2, Nanog, telomerase reverse transcriptase (hTERT), CD9, CD13, CD29, CD44, CD166, DAZL, Runx-1, and a combination thereof.

In some embodiments, the fibroblasts are reprogrammed to possess a more immature phenotype. Immature phenotype in at least some cases is defined as expression of markers associated with pluripotency, these include, for example: a) OCT-4; b) NANOG; c) SOX-2; d) hTERT; and/or e) acetylated histones. The reprograming to a more immature phenotype may comprise a nuclear transfer, a cytoplasmic transfer, contacting the fibroblasts with a DNA methyltransferase inhibitor, contacting the fibroblasts with a histone deacetylase inhibitor, contacting the fibroblasts with a GSK-3 inhibitor, dedifferentiation by alteration of extracellular conditions (alteration of extracellular conditions may include culturing cells in pH lower that 7 but higher than 5 and/or culturing cells in hypoxia and/or culturing in media conditioned by stem cells, such as pluripotent stem cells), or a combination thereof. The nuclear transfer may comprise introducing nuclear material to a cell, such as a fibroblast, that is substantially enucleated. The nuclear material may be derived from a host whose genetic profile is sought to be dedifferentiated, and the cells may include cells expressing one or more of the following: a) OCT-4; b) NANOG; c) SOX-2; d) hTERT; e) acetylated histones; and f) a combination thereof. Specific examples of cells include stem cells derived from parthenogenic reprogramming, iPS cells, stem cells generated from somatic cell nuclear transfer, and embryonic stem cells. In some embodiments, the cytoplasmic transfer comprises introducing cytoplasmic contents from a cell with a dedifferentiated phenotype into a cell with a differentiated phenotype, wherein the cell with a differentiated phenotype (differentiated phenotype may include cells that do not express: a) OCT-4; b) NANOG; c) SOX-2; d) hTERT; e) acetylated histones; or f) a combination thereof) substantially reverts to a dedifferentiated phenotype. In some embodiments, the DNA demethylating agent comprises 5-azacytidine, psammaplin A, zebularine, or a combination thereof. In some embodiments, the histone deacetylase inhibitor comprises valproic acid, trichostatin-A, trapoxin A, depsipeptide, or a combination thereof.

In some embodiments, the fibroblasts are selected from cells in a side population. The side population of cells may be identified based on expression multidrug resistance transport protein (ABCG2) or ability to efflux intracellular dyes such as rhodamine-123 and or Hoechst 33342. In some embodiments, the side population cells are derived from tissues such as pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In some embodiments, the fibroblasts are selected from blood, including from mobilized blood. The mobilized blood may be blood that has been administered a mobilizing agent. The mobilizing agent may be any suitable composition including agents selected from the group consisting of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, and a combination thereof. In certain embodiments, the mobilization therapy comprises a therapy selected from the group consisting of exercise, hyperbaric oxygen, autohemotherapy by ex vivo ozonation of peripheral blood, induction of SDF-1 secretion in an anatomical area outside of the bone marrow, and a combination thereof. In at least some cases, a goal of mobilization therapy is to cause stem cells residing in the bone marrow to exit the bone marrow and enter systemic circulation.

In some embodiments, an antioxidant is administered at a therapeutically effective concentration to the individual. The antioxidant may be any composition with antioxidant activity, including compositions selected from the group consisting of ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, superoxide dismutase, and a combination thereof. The antioxidant may be administered prior to, concurrently with, and/or subsequent to the administration of fibroblasts and/or fibroblast-derived products. Administration of an antioxidant prior to administration of fibroblasts and/or fibroblast-derived products may reduce an inhibitory effect of oxidative stress on the fibroblast therapeutic activity. Administration of an antioxidant(s) concurrently with the administration of fibroblasts and/or fibroblast-derived products may allow maximum fibroblast therapeutic activity; in specific embodiments, the antioxidant(s) is administered at substantially the same time as the administration of fibroblasts and/or fibroblast-derived products.

Certain embodiments concern the modulation of complement activity in an individual. In some embodiments, the modulation of complement activity comprises administering to the individual at least one composition that inhibits the formation of terminal complement complex or C5a (a complement of the complement cascade). The composition that inhibits the formation of terminal complement complex or C5a may comprise a whole antibody or an antibody fragment, which may be a human, humanized, chimerized, or deimmunized antibody or antibody fragment. The whole antibody or antibody fragment may inhibit the cleavage of complement C5. In some embodiments, the antibody fragment is selected from the group consisting of an Fab, an F(ab′)₂, an Fv, a domain antibody, a single-chain antibody, and a combination thereof. In some embodiments, the antibody fragment is pexelizumab. In some embodiments, the whole antibody is eculizumab. A therapeutically effective amount of the whole antibody or antibody fragment may be administered to an individual once every day, every 2 days, every 3 days, every 4 days, every 5 days, every 6 days, every week, or every 2 weeks.

In some embodiments, modulating complement activation comprises administering a composition selected from the group consisting of i) soluble complement receptor, ii) CD59, iii) CD55, iv) CD46, v) an antibody to C5, C6, C7, C8, or C9, and vi) a combination thereof.

Fibroblast therapeutic activity may comprise any activity inherent in or acquired by fibroblasts or fibroblasts-derived products. In some embodiments, fibroblast therapeutic activity comprises an ability of the fibroblast and/or fibroblast-derived products to home to an area of tissue injury. Tissue injury may comprise activation of the coagulation cascade, loss of mitochondrial activity, activation of tissue factor expression(by mRNA using RT-PCR, by protein expression using flow cytometry), induction of ischemia, reduction in ATP usage (for example, as measured by mass spectrometry), reduction in the release of tissue adenosine, or a combination thereof. In some embodiments, the ability of the fibroblasts and/or fibroblast-derived products to home to an area of tissue is facilitated by an increased expression of at least one homing receptor (that is, proteins that detect chemotactic cytokines; as one example, CXCR4 is a homing receptor that recognizes SDF-1 generated by injured tissue). The homing receptor may comprise CXCR-4, CCR-5, VEGF receptor 2 (VEGFR2), or a combination thereof.

In some embodiments, fibroblast therapeutic activity comprises an ability to suppress a pathological immune response. The pathological immune response may comprise tissue destruction, loss of function, and/or fibrosis. In some embodiments, the pathological immune response is associated with production of interleukin-17 (IL-17). IL-17 production may be mediated by an increased number of Th17 cells and/or an increased activity of Th17 cells. The pathological immune response may comprise neutrophil activation, a reduction in neutrophil apoptosis, and/or macrophage activation. Macrophage activation may comprise an increase of nitric oxide and/or oxygen free radicals released by macrophages. Macrophage activation may comprise an increased production of matrix metalloproteases and/or exposure of immunogenic epitopes that activate natural antibodies. The macrophages may comprise M1 macrophages.

Embodiments of the disclosure include methods of increasing fibroblast therapeutic activity or the therapeutic activity of and/or fibroblast derived products for or in an individual, comprising the steps of: optionally assessing a potential for complement activation in an individual; modulating complement activation in the individual; and administering a therapeutically effective amount of fibroblasts and/or fibroblast derived products to the individual. In some cases, the fibroblasts are derived from tissues selected from the group consisting of cord blood, Wharton's Jelly, placenta, bone marrow, adipose tissue, skin, deciduous teeth, nails, peripheral blood, omentum, and a combination thereof. The fibroblasts may be allogeneic, autologous, or xenogeneic with respect to the individual. The fibroblast-derived products may comprise exosomes derived from fibroblasts. Any fibroblasts may be plastic adherent. In some cases, the fibroblasts express markers selected from the group consisting of CD73, CD105, stage-specific embryonic antigens (SSEA) 3, SSEA 4, Tra-1-60, Tra-1-81, Oct-3/4, Cripto, gastrin-releasing peptide (GRP) receptor, podocalyxin-like protein (PODXL), Rex-1, GCTM-2, Nanog, telomerase reverse transcriptase (hTERT), CD9, CD13, CD29, CD44, CD166, DAZL, Runx-1, and a combination thereof.

In some cases, the fibroblasts are reprogrammed to possess a more immature phenotype than fibroblasts that are not reprogrammed. Reprogrammed fibroblasts may express one or more pluripotency markers, such as comprising a) OCT-4; b) NANOG; c) SOX-2; d) hTERT; e) acetylated histones; and f) a combination thereof. In some embodiments, the reprogramming comprises a nuclear transfer, a cytoplasmic transfer, contacting the fibroblasts with one or more DNA methyltransferase inhibitors, contacting the fibroblasts with one or more histone deacetylase inhibitors, contacting the fibroblasts with one or more GSK-3 inhibitors, dedifferentiation by alteration of one or more extracellular conditions (culturing the fibroblasts in a pH from 5-7, culturing the fibroblasts in hypoxia, and/or culturing the fibroblasts in media conditioned by pluripotent stem cells), or a combination thereof. Nuclear transfer may comprise introducing nuclear material to a substantially enucleated cell, such as an oocyte. In cases of cytoplasmic transfer, the cytoplasmic transfer may comprise introducing cytoplasmic contents from a cell with a dedifferentiated phenotype into a cell with a differentiated phenotype, wherein the cell with a differentiated phenotype substantially reverts to a dedifferentiated phenotype. Cells with a differentiated phenotype are fibroblasts, in specific cases. The DNA demethylating agent may comprise 5-azacytidine, psammaplin A, zebularine, or a combination thereof. The histone deacetylase inhibitor may comprise valproic acid, trichostatin-A, trapoxin A, depsipeptide, or a combination thereof.

In some embodiments, the fibroblasts are selected from cells in a side population, and the side population cells may be identified based on expression of multidrug resistance transport protein (ABCG2) and/or an ability to efflux one or more intracellular dyes (e.g., rhodamine-123 and/or Hoechst 33342). The side population cells may be derived from tissues selected from the group consisting of pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

In some embodiments, fibroblasts are obtained or enriched from blood, including at least mobilized blood that may be blood derived from blood that has been administered one or more mobilizing agents and/or blood from an individual that has been administered one or more mobilizing agents. Examples of mobilizing agents may beselected from the group consisting of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, one or more small molecule antagonists of SDF-1, and a combination thereof. In some cases, an individual is provided an effective amount of one or more mobilization therapies comprises a therapy selected from the group consisting of exercise, hyperbaric oxygen, autohemotherapy by ex vivo ozonation of peripheral blood, induction of SDF-1 secretion in an anatomical area outside of the bone marrow, and a combination thereof.

In some embodiments, one or more antioxidants are administered at a therapeutically sufficient concentration or otherwise effective amount to the individual. The antioxidant may be selected from the group consisting of ascorbic acid or one or more derivatives thereof, alpha tocopherol or one or more derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, superoxide dismutase, and a combination thereof. In some embodiments, the antioxidant is administered prior to administration of fibroblasts and/or fibroblast-derived products at a concentration sufficient to reduce an inhibitory effect of oxidative stress on the fibroblast therapeutic activity. In some cases, the antioxidant is administered concurrently with the fibroblasts and/or fibroblast-derived products or subsequent to the fibroblasts and/or fibroblast-derived products cell administration. In some embodiments, modulating complement activation comprises administering to the individual at least one composition that inhibits the formation of terminal complement complex or C5a. In certain aspects, at least one of the compositions that inhibits the formation of terminal complement complex or C5a comprises a whole antibody or an antibody fragment, including comprising a human, humanized, chimerized, or deimmunized antibody or antibody fragment. In at least some cases, the whole antibody or antibody fragment inhibits cleavage of complement C5. The antibody fragment may be selected from the group consisting of an Fab, an F(ab′)2, an Fv, a domain antibody, a single-chain antibody, and a combination thereof. In specific cases, the antibody fragment is pexelizumab and/or the whole antibody is eculizumab. In specific aspects, a therapeutically effective amount of eculizumab is administered to the individual once every 2 weeks. The modulating complement activation may comprise administering a composition selected from the group consisting of i) soluble complement receptor, ii) CD59, iii) CD55, iv) CD46, v) an antibody to C5, C6, C7, C8, or C9, and vi) a combination thereof.

In some embodiments, the fibroblast therapeutic activity comprises an ability of the fibroblast and/or fibroblast-derived products to home to an area of tissue injury in the individual. The tissue injury may comprise activation of the coagulation cascade. The tissue injury may comprise loss of mitochondrial activity, such as is measured as the ability to generate ATP. The tissue injury may comprise activation of tissue factor expression in cells of the tissue. In at least some cases, tissue injury comprises induction of ischemia; tissue injury comprises reduction in ATP usage in damaged tissue cells; and/or tissue injury comprises reduction in release of tissue adenosine. In specific embodiments, the ability of the fibroblast and/or fibroblast-derived products to home is facilitated by an increased expression of at least one homing receptor in the fibroblasts, such as CXCR-4, CCR-5, or VEGF receptor 2. In certain embodiments, the fibroblast therapeutic activity comprises an ability to suppress a pathological immune response in the individual. The pathological immune response may comprise tissue destruction, loss of function, and/or fibrosis. The pathological immune response may be associated with production of interleukin-17, and the production of interleukin-17 may be mediated by an increased number of Th17 cells and/or increased activity of Th17 cells. In certain embodiments, the pathological immune response comprises neutrophil activation in the individual, reduction in neutrophil apoptosis, and/or macrophage activation (which may comprise an increase of nitric oxide and/or oxygen free radicals released by macrophages, including M1 macrophages). In some cases, the macrophage activation comprises increased production of matrix metalloproteases and/or exposure of one or more immunogenic epitopes that activate natural antibodies.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the FIGURES is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIG. 1 shows prolongation of fibroblast survival in blood by complement depletion. Percent viability was measured via an MTT assay in fibroblasts cultured in media (control), heparinized blood, or decomplemented blood for 0, 2, 4, or 8 hours. Control, Blood, and Decomplemented Blood are presented from left to right in the grouped bars.

### DETAILED DESCRIPTION

### I. Definitions

Before the present disclosure is described in detail below, it is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art to which this invention belongs.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, “fibroblast therapeutic activity” refers to activities, functions, and/or properties of fibroblasts. Fibroblast therapeutic activity may also refer to activities, functions, and/or properties of fibroblast-derived products.

As used herein, “side population” refers to a sub-population of cells that is distinct from the main population of cells on the basis of the markers employed. The sub-population of cells may have a distinct expression pattern of markers from the main population, as measured by flow cytometry.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

An “epitope”, also known as antigenic determinant, is the part of a macromolecule that is recognized by the immune system, specifically by antibodies, B cells, or T cells. As used herein, an “epitope” is the part of a macromolecule capable of binding to a compound (e.g. an antibody or antigen-binding fragment thereof) as described herein. In this context, the term “binding” preferably relates to a specific binding. Epitopes usually consist of chemically active surface groupings of molecules such as amino acids or sugar side chains and usually have specific three-dimensional structural characteristics, as well as specific charge characteristics. Conformational and non-conformational epitopes can be distinguished in that the binding to the former but not the latter is lost in the presence of denaturing solvents.

As used herein, “inhibitor of C5a” refers to a compound that inhibits a biological activity of C5a. The term “inhibitor of C5a” particularly refers to a compound that interferes with the binding of C5a to the C5a receptors, C5aR and C5L2; especially to a compound that interferes with the binding of C5a to C5aR. Accordingly, the term “inhibitor of C5a” encompasses compounds that specifically bind to C5a and inhibit binding of C5a to C5aR as well as compounds that specifically bind to C5aR and inhibit binding of C5a to C5aR. Exemplary inhibitors of C5a include the C5a inhibitory peptide (C5aIP), the selective C5a receptor antagonists PMX53 and CCX168, and the anti-05a antibodies disclosed in WO 2011/063980 A1 (also published as US 2012/0231008 A1). The term “inhibitor of C5a” and “C5a inhibitor” are used interchangeably herein.

As used herein, “antibody” typically refers to a glycoprotein comprising at least two heavy (H) chains and two light (L) chains inter-connected by disulfide bonds, or an antigen-binding portion thereof. The term “antibody” also includes all recombinant forms of antibodies, in particular of the antibodies described herein, e.g. antibodies expressed in prokaryotes, unglycosylated antibodies, antibodies expressed in eukaryotes (e.g. CHO cells), glycosylated antibodies, and any antigen-binding antibody fragments and derivatives as described below. Each heavy chain is comprised of a heavy chain variable region (abbreviated herein as VH or V_{H}) and a heavy chain constant region (abbreviated herein as CH or C_{H}). The heavy chain constant region can be further subdivided into three parts, referred to as CH1, CH2, and CH3 (or C_{H}1, C_{H}2, and C_{H}3). Each light chain is comprised of a light chain variable region (abbreviated herein as VL or V_{L}) and a light chain constant region (abbreviated herein as CL or CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “patient”, “subject”, or “individual” may be used interchangeably. The terms refer to any mammal or bird who may benefit from a treatment, such as with any inhibitor of C5a described herein. A patient may be selected from the group consisting of laboratory animals (e.g. mouse or rat), domestic animals (including e.g. guinea pig, rabbit, chicken, turkey, pig, sheep, goat, camel, cow, horse, donkey, cat, or dog), or primates including monkeys (e.g. African green monkeys, chimpanzees, bonobos, gorillas) and human beings.

As used herein, “treat”, “treating”, or “treatment” of a disease or disorder means, for example, accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a particular carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

When referring to cultured vertebrate cells, the term “senescence” (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their non-dividing state for as long as three years. These cells are very much alive and metabolically active, but they do not divide. The non-dividing state of senescent cells has not yet been found to be reversible by any biological, chemical, or viral agent.

As used herein, the term “Growth Medium” generally refers to a medium sufficient for the culturing of cells. In particular embodiments, medium for the culturing of the cells herein comprises Dulbecco's Modified Essential Media (also abbreviated DMEM herein). DMEM-low glucose may be used (also DMEM-LG herein) (Invitrogen, Carlsbad, Calif.). The DMEM-low glucose may be supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some embodiments, different growth media are used, or different supplementations are provided, and these are normally indicated in the text as supplementations to Growth Medium.

### II. Methods and Compositions for Augmenting Fibroblast Therapeutic Activity

The disclosure provides mean of enhancing fibroblast cellular therapy efficacy by inhibition of complement activation. Certain embodiments concern the reduction of complement activation through inhibition the classical, alternative or other pathways as a means of augmenting efficacy of fibroblast therapy. Particular embodiments teach the enhancement of fibroblast cell viability in vivo by complement depletion using agents such as cobra venom factor and/or antibodies to complement components such as C3 and/or C5. Other embodiments of the disclosure comprise maintaining therapeutic activity of fibroblasts in vivo by administration of complement inhibitors. In certain embodiments, the inhibition of complement activity is caused by chronic administration of one or more drugs directed against complement C5. An example of a drug that inhibits complement activity is an antibody specific to one or more components of complement, for example, C5. In certain embodiments, the antibody inhibits the cleavage of C5 and thereby inhibits the formation of both C5a and C5b-9. The antibody may be, e.g., a monoclonal antibody, a chimeric antibody (e.g., a humanized antibody), an antibody fragment (e.g., Fab), a single chain antibody, an Fv, or a domain antibody. Other inhibitors of complement include various agents that are known to those of skill in the art. Antibodies can be made to individual components of activated complement, e.g., antibodies to C5a, C7, C9, etc. (see, e.g., U.S. Pat. No. 6,534,058; published U.S. patent application US 2003/0129187; and U.S. Pat. No. 5,660,825). Proteins are known which inhibit complement-mediated lysis, including CD59, CD55, CD46 and other inhibitors of C8 and C9 (see, e.g., U.S. Pat. No. 6,100,443). U.S. Pat. No. 6,355,245 teaches an antibody which binds to C5 and prevents it from being cleaved into C5a and C5b thereby preventing the formation not only of C5a but also the C5b-9 complex. Proteins known as complement receptors and which bind complement are also known (see, Published PCT Patent Application WO 92/10205 and U.S. Pat. No. 6,057,131). Use of soluble forms of complement receptors, e.g., soluble CR1, can inhibit the consequences of complement activation such as neutrophil oxidative burst, complement mediated hemolysis, and C3a and C5a production. Those of skill in the art recognize the above as some, but not all, of the known methods of inhibiting complement and its activation.

In some embodiments, methods for enhancing fibroblast therapeutic activity comprise the use of RNA interference in order to suppress expression of the C5 gene, including in fibroblasts and/or systemic tissue, including at least the liver (a major source of the protein). Previous publications have described the use of siRNA and shRNA in order to suppress C5 gene expression and are incorporated by reference [33, 107-113]. In particular embodiments, suppression of C5 is achieved by administering a double-stranded ribonucleic acid (dsRNA) composition for inhibiting expression of complement component C5. The dsRNA agent may comprise a sense strand and an antisense strand. In some embodiments, the sense strand comprises the nucleotide sequence AAGCAAGAUAUUUUUAUAAUA (SEQ ID NO:1). In some embodiments, the antisense strand comprises the nucleotide sequence UAUUAUAAAAAUAUCUUGCUUUU (SEQ ID NO:2). In one embodiment, the dsRNA composition comprises at least one modified nucleotide, as described below. In particular embodiments, the present disclosure provides a double stranded RNAi agent for inhibiting expression of complement component C5 wherein the double stranded RNAi agent comprises a sense strand and an antisense strand forming a double-stranded region, wherein the sense strand comprises at least 15 contiguous nucleotides differing by no more than 3 nucleotides wherein substantially all of the nucleotides of the sense strand and substantially all of the nucleotides of the antisense strand are modified nucleotides, and wherein the sense strand is conjugated to a ligand attached at the 3′-terminus.

In one embodiment, all of the nucleotides of the sense strand and all of the nucleotides of the antisense strand comprise a modification. In one embodiment, substantially all of the nucleotides of the sense strand are modified nucleotides selected from the group consisting of a 2′-O-methyl modification, a 2′-fluoro modification and a 3′-terminal deoxy-thymine (dT) nucleotide. In another embodiment, substantially all of the nucleotides of the antisense strand are modified nucleotides selected from the group consisting of a 2′-O-methyl modification, a 2′-fluoro modification and a 3′-terminal deoxy-thymine (dT) nucleotide. In another embodiment, the modified nucleotides are a short sequence of deoxy-thymine (dT) nucleotides. In another embodiment, the sense strand comprises two phosphorothioate internucleotide linkages at the 5′-terminus. In one embodiment, the antisense strand comprises two phosphorothioate internucleotide linkages at the 5′-terminus and two phosphorothioate internucleotide linkages at the 3′-terminus. In yet another embodiment, the sense strand is conjugated to one or more GalNAc derivatives attached through a branched bivalent or trivalent linker at the 3′-terminus. In one embodiment, substantially all of the nucleotides of the sense strand are modified nucleotides selected from the group consisting of a 2′-O-methyl modification, a 2′-fluoro modification and a 3′-terminal dT nucleotide. In another embodiment, substantially all of the nucleotides of the antisense strand are modified nucleotides selected from the group consisting of a 2′-O-methyl modification, a 2′-fluoro modification and a 3′-terminal dT nucleotide. In another embodiment, the modified nucleotides are a short sequence of deoxy-thymine (dT) nucleotides. In another embodiment, the sense strand comprises two phosphorothioate internucleotide linkages at the 5′-terminus. In one embodiment, the antisense strand comprises two phosphorothioate internucleotide linkages at the 5′-terminus and two phosphorothioate internucleotide linkages at the 3′-terminus. In yet another embodiment, the sense strand is conjugated to one or more GalNAc derivatives attached through a branched bivalent or trivalent linker at the 3′-terminus.

In particular embodiments, the subject is human, and said human is treated with a cell therapy and an anti-complement component C5 antibody, or antigen-binding fragment thereof

In particular embodiments, inhibition of complement activity (such as C5 activity) in an individual is performed together with administration of fibroblasts to the individual. In particular embodiments, fibroblasts comprise cells that are (1) adherent to plastic, (2) express CD73, CD90, and CD105 antigens, while being CD14, CD34, CD45, and HLA-DR negative, and (3) possess an ability to differentiate to osteogenic, chondrogenic and adipogenic lineage.

In some embodiments, fibroblasts are administered to an individual together with an antibody, or antigen-binding fragment thereof, that inhibits cleavage of complement component C5 into fragments C5a and C5b. In particular embodiments, the anti-complement component C5 antibody is eculizumab. In one embodiment, eculizumab is administered to the individual weekly at a dose less than about 600 mg for 4 weeks followed by a fifth dose at about one week later of less than about 900 mg, followed by a dose less than about 900 mg about every two weeks thereafter before, concurrent with, or after fibroblast therapy. In another embodiment, eculizumab is administered to the individual weekly at a dose less than about 900 mg for 4 weeks followed by a fifth dose at about one week later of less than about 1200 mg, followed by a dose less than about 1200 mg about every two weeks thereafter before, concurrent or after fibroblast therapy. In one embodiment, eculizumab is administered to the subject weekly at a dose less than about 900 mg for 4 weeks followed by a fifth dose at about one week later of less than about 1200 mg, followed by a dose less than about 1200 mg about every two weeks thereafter before, concurrent or after fibroblast therapy.

In another embodiment, eculizumab is administered to the individual weekly at a dose less than about 600 mg for 2 weeks followed by a third dose at about one week later of less than about 900 mg, followed by a dose less than about 900 mg about every two weeks thereafter before, concurrent with, or after fibroblast therapy. In another embodiment, eculizumab is administered to the individual weekly at a dose less than about 600 mg for 2 weeks followed by a third dose at about one week later of less than about 600 mg, followed by a dose less than about 600 mg about every two weeks thereafter before, concurrent with, or after fibroblast therapy. In yet another embodiment, eculizumab is administered to the individual weekly at a dose less than about 600 mg for 1 week followed by a second dose at about one week later of less than about 300 mg, followed by a dose less than about 300 mg about every two weeks thereafter before, concurrent with, or after fibroblast therapy. In one embodiment, eculizumab is administered to the individual weekly at a dose less than about 300 mg for 1 week followed by a second dose at about one week later of less than about 300 mg, followed by a dose less than about 300 mg about every two weeks thereafter before, concurrent with, or after fibroblast therapy. Certain embodiments further include plasmapheresis or plasma exchange in the individual. In one such embodiment, eculizumab is administered to the individual at a dose less than about 600 mg or at a dose less than about 300 mg before, concurrent with, or after fibroblast therapy. Certain methods include plasma infusion in the individual. In one such embodiment, eculizumab is administered to the individual at a dose less than about 300 mg before, concurrent with, or after fibroblast therapy. In one embodiment, eculizumab is administered to the individual at a dose of about 0.01 mg/kg to about 10 mg/kg or about 0.5 mg/kg to about 15 mg/kg. In another embodiment, eculizumab is administered to the individual at a dose of about 5 mg/kg to about 15 mg/kg before, concurrent with, or after fibroblast therapy. In one embodiment, eculizumab is administered to the individual at a dose selected from the group consisting of 0.5 mg/kg, 1 mg/kg, 1.5 mg/kg, 3 mg/kg, 5 mg/kg, 7 mg/kg, 10 mg/kg, and 15 mg/kg before, concurrent with, or after fibroblast therapy. In one embodiment, eculizumab is administered to the individual via an intravenous infusion. In another embodiment, eculizumab is administered to the individual subcutaneously.

Certain embodiments concern the uses of one or more drugs that inhibit complement activity and immunosuppressive agents in the manufacture of a medicament or medicament package. Such medicament or medicament package is useful in enhancing therapeutic activity of cell therapies. In certain embodiments, the medicament or medicament package increases activity of fibroblast cell therapy. In certain embodiments, the medicament or medicament package increases fibroblast therapeutic activity. In some embodiments, the medicament or medicament package is formulated and prepared such that it is suitable for chronic administration. In some embodiments, stable formulations are employed. In certain embodiments, the medicament or medicament package is formulated and prepared such that it is suitable for concurrent administration of the drug that inhibits complement activity and the immunosuppressive drug to the recipient individual. In certain embodiments, the medicament or medicament package is formulated and prepared such that it is suitable for sequential (in either order) administration of the drug that inhibits complement activity and the immunosuppressive drug to the recipient individual.

In certain embodiments, fibroblasts are used together with complement inhibition for the purpose of immune modulation. The invention discloses that fibroblasts may be viewed as a “intelligent” immune modulators. In contrast to current therapies, which globally cause immune suppression, production of anti-inflammatory factors by fibroblasts appears to be dependent on their environment, with upregulation of factors such as TGF-b, HLA-G, IL-10, and neuropilin-A ligands galectin-1 and Semaphorin-3A in response to immune/inflammatory stimuli but little in the basal state. These properties may be selected for when utilizing the marker combinations disclosed in the current invention. Additionally, the invention discloses synergies between complement inhibition and fibroblast administration of induction of immune modulation and/or tolerogenesis. The combined use of fibroblasts and complement inhibition may be directed towards any known use of fibroblasts, including towards conditions such as autoimmunity, transplant rejection, inflammation, sepsis, ARDS and acute radiation syndrome.

Additionally, systemically administered fibroblasts possess ability to selectively home to injured/hypoxic areas by recognition of signals such as HMGB1 or CXCR1, respectively. The ability to home to injury, combined with selective induction of immune modulation only in response to inflammatory/danger signals suggests the possibility that systemically administered fibroblasts do not cause global immune suppression.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In some embodiments, cells of the disclosure are cultured at 37° C., in a standard atmosphere comprising 5% CO₂. Relative humidity may be maintained at about 100%. While foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

### III. Administration

The therapy provided herein may comprise administration of a therapeutic agents (e.g., fibroblasts and/or fibroblast-derived products, modulators of complement, and the like) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. The therapeutic agents (e.g., fibroblasts) of the disclosure may be administered by the same route of administration or by different routes of administration. In some embodiments, the fibroblasts and/or fibroblast-derived products are administered intravenously, intramuscularly, subcutaneously, topically, orally, transdermally, intraperitoneally, intraorbitally, by implantation, by inhalation, intrathecally, intraventricularly, or intranasally. In some embodiments, the modulator of complement is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, 200, 300, 400, 500, or 1000 μg/kg, mg/kg, μg/day, or mg/day or any range derivable therein. Furthermore, such doses can be administered at multiple times during a day, and/or on multiple days, weeks, or months.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

In some embodiments, a pharmaceutical composition (including any described herein), such as an inhibitor of C5a (e.g. a binding moiety specifically binding to C5a, especially hC5a, as described herein) may be administered to a patient by any route established in the art which provides a sufficient level of the inhibitor of C5a in the patient. It can be administered systemically or locally. Such administration may be parenterally, transmucosally, e.g., orally, nasally, rectally, intravaginally, sublingually, submucosally, transdermally, or by inhalation. Parenteral administration includes intravenous or intraperitoneal injection, and also includes, but is not limited to, intra-arterial, intramuscular, intradermal and subcutaneous administration. If the pharmaceutical composition of the present invention is administered locally it can be injected directly into the organ or tissue to be treated.

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient may be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal administration may comprise solid carriers such as powders (preferably having a particle size in the range of 20 to 500 microns). Powders may be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In certain embodiments, pharmaceutical compositions of the invention are administered via the nasal cavity to the lungs.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets.

In certain embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

In another embodiment, a drug, such as the one or more C5a inhibitors encompassed herein, is delivered in a controlled-release system. For example, the inhibitor may be administered using intravenous infusion, an implantable osmotic pump, a transdermal patch, liposomes, or other modes of administration. In one embodiment, a pump may be used. In another embodiment, the compound can be delivered in a vesicle, in particular a liposome (WO 91/04014; U.S. Pat. No. 4,704,355). In another embodiment, polymeric materials can be used

In a specific embodiment, it may be desirable to administer the pharmaceutical compositions including the one or more C5a inhibitors locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

Selection of the particular effective dose will be determined by a skilled artisan based upon considering several factors which will be known to one of ordinary skill in the art. Such factors include the particular form of the pharmaceutical composition, e.g. polypeptide or vector, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be prevented and/or treated or the benefit to be achieved in a normal individual, the body mass of the patient, the patient's age, the route of administration, whether administration is acute or chronic, concomitant medications, and other factors well known to affect the efficacy of administered pharmaceutical agents. Thus, the precise dosage should be decided according to the judgment of the practitioner and each patient's circumstances, e.g. depending upon the condition and the immune status of the individual patient, and according to standard clinical techniques.

### IV. Kits of the Disclosure

Any of the cellular and/or non-cellular compositions described herein or similar thereto may be comprised in a kit. In a non-limiting example, one or more reagents for use in methods for preparing fibroblasts, fibroblast-derived products, or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

A pharmaceutical package of the present disclosure may comprise one or more fibroblast therapies (including fibroblasts and/or fibroblast-derived products) and one or more compositions that inhibits complement activity. The pharmaceutical package may further comprise a label for chronic administration. The pharmaceutical package may also comprise a label for self-administration by an individual, for example, a recipient of a cellular therapy, or instructions for a caretaker of a recipient of cellular therapy. In certain embodiments, the drug and the agent in the pharmaceutical package are in a formulation or separate formulations that are suitable for chronic administration and/or self-administration. The present disclosure also provides lyophilized formulations and formulations suitable for injection. Certain embodiments provide a lyophilized antibody formulation comprising an antibody that inhibits complement activity and a lyoprotectant. In certain embodiments, the antibody formulation is suitable for chronic administration, for example, the antibody formulation stable. Alternative embodiments provide an injection system comprising a syringe; the syringe comprises a cartridge containing an antibody that inhibits complement activity and is in a formulation suitable for injection. An antibody employed in various embodiments of the present disclosure preferably inhibits the formation of terminal complement or C5a. In certain embodiments, antibody inhibits formation of terminal complement or C5a is a whole antibody or an antibody fragment. The whole antibody or antibody fragment may be a human, humanized, chimerized or deimmunized antibody or antibody fragment. In certain embodiments, the whole antibody or antibody fragment may inhibit cleavage of complement C5. In certain embodiments, the antibody fragment is a Fab, an F(ab′)2, an Fv, a domain antibody, or a single-chain antibody. In some embodiments, the antibody fragment is pexelizumab. In some embodiments, the whole antibody is eculizumab.

In certain embodiments, one or more compositions (including one or more antibodies) that inhibits complement activity is present in unit dosage form, which can be particularly suitable for self-administration. Similarly, an immunosuppressive agent of the present disclosure may also be present in unit dosage form. A formulated product of the present disclosure can be included within a container, typically, for example, a vial, cartridge, prefilled syringe or disposable pen. A doser such as the doser device described in U.S. Pat. No. 6,302,855 may also be used, for example, with an injection system of the present disclosure.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Prolongation of Fibroblast Survival in Blood by Complement Depletion

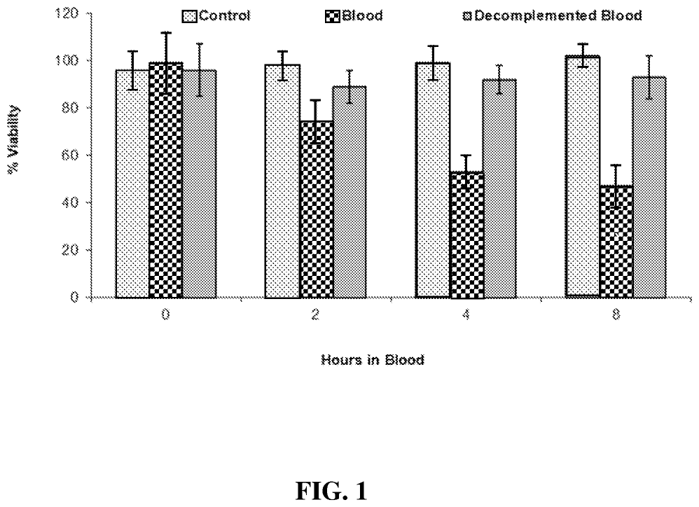

Fibroblasts from foreskin (ATCC) plated to 75% confluence and allowed to adhere overnight on 96 well plates with flat bottoms. Cells had been incubated in DMEM with 10% fetal calf serum. After overnight incubation, media was aspirated off cells and 50 microliters of either culture media (control), heparinized human blood, or heparinized human blood pretreated for 10 minutes with 5 ug/ml of cobra venom factor was added to the cell. Cells were assayed for viability at time 0, 2, 4, and 8 hours. Viability was assessed by adding 50 μl 0·5% MTT was added to each well and after an additional 4-hr incubation, the plate was centrifuged at 500 g for 10 min and the supernatant was discarded. The cells in each well were lysed with 100 μl/well isopropanol—HCl in a VARI shaker for 5 min. The optical density was measured at 570 nm with an ELISA reader. As shown in FIG. 1, complement inhibition of cobra venom factor maintained fibroblast viability in blood.

### REFERENCES

1. Walport, M. J., Complement. First of two parts. N Engl J Med, 2001. 344(14): p. 1058-66.
2. Snyderman, R., H. Gewurz, and S. E. Mergenhagen, Interactions of the complement system with endotoxic lipopolysaccharide. Generation of a factor chemotactic for polymorphonuclear leukocytes. J Exp Med, 1968. 128(2): p. 259-75.
3. Haddad, A. and A. M. Wilson, Biochemistry, Complement, in StatPearls. 2020: Treasure Island (FL).
4. Lehnardt, S., Innate immunity and neuroinflammation in the CNS: the role of microglia in Toll-like receptor-mediated neuronal injury. Glia, 2010. 58(3): p. 253-63.
5. Dunkelberger, J. R. and W. C. Song, Complement and its role in innate and adaptive immune responses. Cell Res, 2010. 20(1): p. 34-50.
6. Morgan, E. L., et al., Anaphylatoxin-mediated regulation of the immune response. II. C5a-mediated enhancement of human humoral and T cell-mediated immune responses. J Immunol, 1983. 130(3): p. 1257-61.
7. Nataf, S., et al., Human T cells express the C5a receptor and are chemoattracted to C5a. J Immunol, 1999. 162(7): p. 4018-23.
8. Marsh, J. E., et al., The allogeneic T and B cell response is strongly dependent on complement components C3 and C4. Transplantation, 2001. 72(7): p. 1310-8.
9. Kim, A. H., et al., Complement C5a receptor is essential for the optimal generation of antiviral CD8+ T cell responses. J Immunol, 2004. 173(4): p. 2524-9.
10. Fang, C., et al., Complement-dependent enhancement of CD8+ T cell immunity to lymphocytic choriomeningitis virus infection in decay-accelerating factor-deficient mice. J Immunol, 2007. 179(5): p. 3178-86.
11. Wang, H., et al., Inhibition of terminal complement components in presensitized transplant recipients prevents antibody-mediated rejection leading to long-term graft survival and accommodation. J Immunol, 2007. 179(7): p. 4451-63.
12. Lalli, P. N., et al., Decay accelerating factor can control T cell differentiation into IFN-gamma-producing effector cells via regulating local C5a-induced IL-12 production. J Immunol, 2007. 179(9): p. 5793-802.
13. Strainic, M. G., et al., Locally produced complement fragments C5a and C3a provide both costimulatory and survival signals to naive CD4+ T cells. Immunity, 2008. 28(3): p. 425-35.
14. Liu, J., et al., IFN-gamma and IL-17 production in experimental autoimmune encephalomyelitis depends on local APC-T cell complement production. J Immunol, 2008. 180(9): p. 5882-9.
15. Hegde, G. V., et al., A conformationally-biased, response-selective agonist of C5a acts as a molecular adjuvant by modulating antigen processing and presentation activities of human dendritic cells. Int Immunopharmacol, 2008. 8(6): p. 819-27.
16. Lalli, P. N., et al., Locally produced C5a binds to T cell-expressed C5aR to enhance effector T-cell expansion by limiting antigen-induced apoptosis. Blood, 2008. 112(5): p. 1759-66.
17. Pavlov, V., et al., Donor deficiency of decay-accelerating factor accelerates murine T cell-mediated cardiac allograft rejection. J Immunol, 2008. 181(7): p. 4580-9.
18. Fang, C., et al., Complement promotes the development of inflammatory T-helper 17 cells through synergistic interaction with Toll-like receptor signaling and interleukin-6 production. Blood, 2009. 114(5): p. 1005-15.
19. Raedler, H., et al., Primed CD8(+) T-cell responses to allogeneic endothelial cells are controlled by local complement activation. Am J Transplant, 2009. 9(8): p. 1784-95.
20. Li, Q., et al., The complement inhibitor FUT-175 suppresses T cell autoreactivity in experimental autoimmune encephalomyelitis. Am J Pathol, 2009. 175(2): p. 661-7.
21. Li, Q., et al., Augmenting DAF levels in vivo ameliorates experimental autoimmune encephalomyelitis. Mol Immunol, 2009. 46(15): p. 2885-91.
22. Xu, R., et al., Complement C5a regulates IL-17 by affecting the crosstalk between DC and gammadelta T cells in CLP-induced sepsis. Eur J Immunol, 2010. 40(4): p. 1079-88.
23. Hashimoto, M., et al., Complement drives Th17 cell differentiation and triggers autoimmune arthritis. J Exp Med, 2010. 207(6): p. 1135-43.
24. Chen, G., et al., Blockade of complement activation product C5a activity using specific antibody attenuates intestinal damage in trinitrobenzene sulfonic acid induced model of colitis. Lab Invest, 2011. 91(3): p. 472-83.
25. Han, G., et al., gammadeltaT-cell function in sepsis is modulated by C5a receptor signalling. Immunology, 2011. 133(3): p. 340-9.
26. Raedler, H., et al., Anti-complement component C5 mAb synergizes with CTLA4Ig to inhibit alloreactive T cells and prolong cardiac allograft survival in mice. Am J Transplant, 2011. 11(7): p. 1397-406.
27. Vieyra, M., et al., Complement regulates CD4 T-cell help to CD8 T cells required for murine allograft rejection. Am J Pathol, 2011. 179(2): p. 766-74.
28. Fusakio, M. E., et al., C5a regulates NKT and NK cell functions in sepsis. J Immunol, 2011. 187(11): p. 5805-12.
29. Mashruwala, M. A., et al., A defect in the synthesis of Interferon-gamma by the T cells of Complement-05 deficient mice leads to enhanced susceptibility for tuberculosis. Tuberculosis (Edinb), 2011. 91 Suppl 1: p. S82-9.
30. Strainic, M. G., et al., Absence of signaling into CD4(+) cells via C3aR and C5aR enables autoinductive TGF-betal signaling and induction of Foxp3(+) regulatory T cells. Nat Immunol, 2013. 14(2): p. 162-71.
31. Liu, T., et al., An essential role for C5aR signaling in the optimal induction of a malaria-specific CD4+ T cell response by a whole-killed blood-stage vaccine. J Immunol, 2013. 191(1): p. 178-86.
32. Ma, N., et al., C5a regulates IL-12+DC migration to induce pathogenic Th1 and Th17 cells in sepsis. PLoS One, 2013. 8(7): p. e69779.
33. Cravedi, P., et al., Immune cell-derived C3a and C5a costimulate human T cell alloimmunity. Am J Transplant, 2013. 13(10): p. 2530-9.
34. Yamanaka, K., et al., Depression of Complement Regulatory Factors in Rat and Human Renal Grafts Is Associated with the Progress of Acute T-Cell Mediated Rejection. PLoS One, 2016. 11(2): p. e0148881.
35. Steinman, R. M., Dendritic cells: understanding immunogenicity. Eur J Immunol, 2007. 37 Suppl 1: p. S53-60.
36. Levings, M. K., et al., Differentiation of Tr1 cells by immature dendritic cells requires IL-10 but not CD25+CD4+ Tr cells. Blood, 2005. 105(3): p. 1162-9.
37. Ghiringhelli, F., et al., Tumor cells convert immature myeloid dendritic cells into TGF-beta-secreting cells inducing CD4+CD25+ regulatory T cell proliferation. J Exp Med, 2005. 202(7): p. 919-29.
38. Stepkowski, S. M., et al., Immature syngeneic dendritic cells potentiate tolerance to pancreatic islet allografts depleted of donor dendritic cells in microgravity culture condition. Transplantation, 2006. 82(12): p. 1756-63.
39. Jin, Y., et al., Induction of auto-reactive regulatory T cells by stimulation with immature autologous dendritic cells. Immunol Invest, 2007. 36(2): p. 213-32.
40. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.
41. Ureta, G., et al., Generation of dendritic cells with regulatory properties. Transplant Proc, 2007. 39(3): p. 633-7.
42. Yamazaki, S., et al., Dendritic cells are specialized accessory cells along with TGF-for the differentiation of Foxp3+ CD4+ regulatory T cells from peripheral Foxp3 precursors. Blood, 2007. 110(13): p. 4293-302.
43. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.
44. Wu, K., et al., Suppression of allergic inflammation by allergen-DNA-modified dendritic cells depends on the induction of Foxp3+ Regulatory T cells. Scand J Immunol, 2008. 67(2): p. 140-51.
45. Sarris, M., et al., Neuropilin-1 expression on regulatory T cells enhances their interactions with dendritic cells during antigen recognition. Immunity, 2008. 28(3): p. 402-13.
46. Zhang, X., et al., Generation of therapeutic dendritic cells and regulatory T cells for preventing allogeneic cardiac graft rejection. Clin Immunol, 2008. 127(3): p. 313-21.
47. Cools, N., et al., Immunosuppression induced by immature dendritic cells is mediated by TGF-beta/IL-10 double positive CD4+ regulatory T cells. J Cell Mol Med, 2008. 12(2): p. 690-700.
48. Wang, L., et al., Programmed death 1 ligand signaling regulates the generation of adaptive Foxp3+CD4+ regulatory T cells. Proc Natl Acad Sci USA, 2008. 105(27): p. 9331-6.
49. Marguti, I., et al., Expansion of CD4+ CD25+ Foxp3+ T cells by bone marrow-derived dendritic cells. Immunology, 2009. 127(1): p. 50-61.
50. Qadura, M., et al., Reduction of the immune response to factor VIII mediated through tolerogenic factor VIII presentation by immature dendritic cells. J Thromb Haemost, 2008. 6(12): p. 2095-104.
51. Kuo, Y. R., et al., Alloantigen pulsed host dendritic cells induce T-cell regulation and prolong allograft survival in a rat model of hindlimb allotransplantation. J Surg Res, 2009. 153(2): p. 317-25.
52. Bamboat, Z. M., et al., Human liver dendritic cells promote T cell hyporesponsiveness. J Immunol, 2009. 182(4): p. 1901-11.
53. Jin, C. J., et al., All-trans retinoic acid inhibits the differentiation, maturation, and function of human monocyte-derived dendritic cells. Leuk Res, 2010. 34(4): p. 513-20.
54. Zahorchak, A. F., G. Raimondi, and A. W. Thomson, Rhesus monkey immature monocyte-derived dendritic cells generate alloantigen-specific regulatory T cells from circulating CD4+CD127−/lo T cells. Transplantation, 2009. 88(9): p. 1057-64.
55. Kushwah, R., et al., Apoptotic dendritic cells induce tolerance in mice through suppression of dendritic cell maturation and induction of antigen-specific regulatory T cells. J Immunol, 2009. 183(11): p. 7104-18.
56. Dai, H., et al., Programmed death-1 signaling is essential for the skin allograft protection by alternatively activated dendritic cell infusion in mice. Transplantation, 2009. 88(7): p. 864-73.
57. Wang, L., Adaptive Treg generation by DCs and their functional analysis. Methods Mol Biol, 2010. 595: p. 403-12.
58. Schildknecht, A., et al., FoxP3+ regulatory T cells essentially contribute to peripheral CD8+ T-cell tolerance induced by steady-state dendritic cells. Proc Natl Acad Sci USA, 2010. 107(1): p. 199-203.
59. Heng, Y., et al., Adoptive transfer of FTY720-treated immature BMDCs significantly prolonged cardiac allograft survival. Transpl Int, 2010. 23(12): p. 1259-70.
60. Muller, T., et al., Iloprost has potent anti-inflammatory properties on human monocyte-derived dendritic cells. Clin Exp Allergy, 2010. 40(8): p. 1214-21.
61. Kong, W., J. H. Yen, and D. Ganea, Docosahexaenoic acid prevents dendritic cell maturation, inhibits antigen-specific Th1/Th17 differentiation and suppresses experimental autoimmune encephalomyelitis. Brain Behav Immun, 2011. 25(5): p. 872-82.
62. Takeda, M., et al., Oral administration of an active form of vitamin D3 (calcitriol) decreases atherosclerosis in mice by inducing regulatory T cells and immature dendritic cells with tolerogenic functions. Arterioscler Thromb Vasc Biol, 2010. 30(12): p. 2495-503.
63. Stary, G., et al., Glucocorticosteroids modify Langerhans cells to produce TGF-beta and expand regulatory T cells. J Immunol, 2011. 186(1): p. 103-12.
64. Pletinckx, K., et al., Role of dendritic cell maturity/costimulation for generation, homeostasis, and suppressive activity of regulatory T cells. Front Immunol, 2011. 2: p. 39.
65. Wolfle, S. J., et al., PD-L1 expression on tolerogenic APCs is controlled by STAT-3. Eur J Immunol, 2011. 41(2): p. 413-24.
66. Schuler, P. J., et al., Dendritic cell generation and CD4+ CD25high FOXP3+ regulatory t cells in human head and neck carcinoma during radio-chemotherapy. Eur J Med Res, 2011. 16(2): p. 57-62.
67. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
68. Amaral, M. M., et al., Thioperamide induces CD4 CD25 Foxp3 regulatory T lymphocytes in the lung mucosa of allergic mice through its action on dendritic cells. J Asthma Allergy, 2011. 4: p. 93-102.
69. Presicce, P., et al., Myeloid dendritic cells isolated from tissues of SIV-infected Rhesus macaques promote the induction of regulatory T cells. AIDS, 2012. 26(3): p. 263-73.
70. Wang, G. Y., et al., Rapamycin combined with donor immature dendritic cells promotes liver allograft survival in association with CD4(+) CD25(+) Foxp3(+) regulatory T cell expansion. Hepatol Res, 2012. 42(2): p. 192-202.
71. Petzold, C., et al., Targeted antigen delivery to DEC-205(+) dendritic cells for tolerogenic vaccination. Rev Diabet Stud, 2012. 9(4): p. 305-18.
72. Wang, G. Y., et al., Rapamycin combined with allogenic immature dendritic cells selectively expands CD4+CD25+Foxp3+ regulatory T cells in rats. Hepatobiliary Pancreat Dis Int, 2012. 11(2): p. 203-8.
73. Zhou, F., et al., Immune tolerance induced by intravenous transfer of immature dendritic cells via up-regulating numbers of suppressive IL-10(+) IFN-gamma(+)-producing CD4(+) T cells. Immunol Res, 2013. 56(1): p. 1-8.
74. Volchenkov, R., et al., Type 1 regulatory T cells and regulatory B cells induced by tolerogenic dendritic cells. Scand J Immunol, 2013. 77(4): p. 246-54.
75. Zhang, G., et al., Triptolide-conditioned dendritic cells induce allospecific T-cell regulation and prolong renal graft survival. J Invest Surg, 2013. 26(4): p. 191-9.
76. Farias, A. S., et al., Vitamin D3 induces IDO+ tolerogenic DCs and enhances Treg, reducing the severity of EAE. CNS Neurosci Ther, 2013. 19(4): p. 269-77.
77. Gao, X. W., et al., Mechanism of immune tolerance induced by donor derived immature dendritic cells in rat high-risk corneal transplantation. Int J Ophthalmol, 2013. 6(3): p. 269-75.
78. Lindenberg, J. J., et al., IL-10 conditioning of human skin affects the distribution of migratory dendritic cell subsets and functional T cell differentiation. PLoS One, 2013. 8(7): p. e70237.
79. Huang, Y., et al., Increased expression of herpesvirus entry mediator in 1,25-dihydroxyvitamin D3-treated mouse bone marrow-derived dendritic cells promotes the generation of CD4(+)CD25(+)Foxp3(+) regulatory T cells. Mol Med Rep, 2014. 9(3): p. 813-8.
80. Pletinckx, K., et al., Immature dendritic cells convert anergic nonregulatory T cells into Foxp3− IL-10+ regulatory T cells by engaging CD28 and CTLA-4. Eur J Immunol, 2015. 45(2): p. 480-91.
81. Wei, Y., et al., Infusion of dendritic cells carrying donor lymphocytes treated with 8-methoxypsoralen and ultraviolet A light induces CD19+ IL-10+ regulatory B cells and promotes skin allograft survival. Transplant Proc, 2014. 46(10): p. 3641-6.
82. Dong, M., et al., Rapamycin Combined with Immature Dendritic Cells Attenuates Obliterative Bronchiolitis in Trachea Allograft Rats by Regulating the Balance of Regulatory and Effector T Cells. Int Arch Allergy Immunol, 2015. 167(3): p. 177-85.
83. Bergmann, C., et al., Expansion and characteristics of human T regulatory type 1 cells in co-cultures simulating tumor microenvironment. Cancer Immunol Immunother, 2007. 56(9): p. 1429-42.
84. Kaporis, H. G., et al., Human basal cell carcinoma is associated with Foxp3+ T cells in a Th2 dominant microenvironment. J Invest Dermatol, 2007. 127(10): p. 2391-8.
85. Koido, S., et al., In vitro generation of cytotoxic and regulatory T cells by fusions of human dendritic cells and hepatocellular carcinoma cells. J Transl Med, 2008. 6: p. 51.
86. Li, L., et al., Hepatoma cells inhibit the differentiation and maturation of dendritic cells and increase the production of regulatory T cells. Immunol Lett, 2007. 114(1): p. 38-45.
87. Shi, D., et al., Artificial phosphatidylserine liposome mimics apoptotic cells in inhibiting maturation and immunostimulatory function of murine myeloid dendritic cells in response to 1-chloro-2,4-dinitrobenze in vitro. Arch Dermatol Res, 2007. 299(7): p. 327-36.
88. Kranich, J., et al., Follicular dendritic cells control engulfment of apoptotic bodies by secreting Mfge8. J Exp Med, 2008. 205(6): p. 1293-302.
89. Rodriguez-Manzanet, R., et al., T and B cell hyperactivity and autoimmunity associated with niche-specific defects in apoptotic body clearance in TIM-4-deficient mice. Proc Natl Acad Sci USA, 2010. 107(19): p. 8706-11.
90. Frey, B. and U.S. Gaipl, The immune functions of phosphatidylserine in membranes of dying cells and microvesicles. Semin Immunopathol, 2011. 33(5): p. 497-516.
91. Saas, P., et al., Phosphatidylserine-expressing cell by-products in transfusion: A pro-inflammatory or an anti-inflammatory effect? Transfus Clin Biol, 2012. 19(3): p. 90-7.
92. Trahtemberg, U. and D. Mevorach, Apoptotic Cells Induced Signaling for Immune Homeostasis in Macrophages and Dendritic Cells. Front Immunol, 2017. 8: p. 1356.
93. Perruche, S., et al., CD3-specific antibody-induced immune tolerance involves transforming growth factor-beta from phagocytes digesting apoptotic T cells. Nat Med, 2008. 14(5): p. 528-35.
94. Dumitriu, L E., et al., Human dendritic cells produce TGF-beta 1 under the influence of lung carcinoma cells and prime the differentiation of CD4+CD25+Foxp3+ regulatory T cells. J Immunol, 2009. 182(5): p. 2795-807.
95. Ragni, M. V., et al., Factor VIII-pulsed dendritic cells reduce anti factor VIII antibody formation in the hemophilia A mouse model. Exp Hematol, 2009. 37(6): p. 744-54.
96. Kushwah, R., et al., Uptake of apoptotic DC converts immature DC into tolerogenic DC that induce differentiation of Foxp3+ Treg. Eur J Immunol, 2010. 40(4): p. 1022-35.
97. Zheng, D. H., et al., Uptake of donor lymphocytes treated with 8-methoxypsoralen and ultraviolet A light by recipient dendritic cells induces CD4+CD25+Foxp3+ regulatory T cells and down-regulates cardiac allograft rejection. Biochem Biophys Res Commun, 2010. 395(4): p. 540-6.
98. Carrascal, M. A., et al., Sialyl Tn-expressing bladder cancer cells induce a tolerogenic phenotype in innate and adaptive immune cells. Mol Oncol, 2014. 8(3): p. 753-65.
99. Lutz, M. B., Induction of CD4(+) Regulatory and Polarized Effector/helper T Cells by Dendritic Cells. Immune Netw, 2016. 16(1): p. 13-25.
100. Verbovetski, I., et al., Opsonization of apoptotic cells by autologous iC3b facilitates clearance by immature dendritic cells, down-regulates DR and CD86, and up-regulates CC chemokine receptor 7. J Exp Med, 2002. 196(12): p. 1553-61.
101. Fadok, V. A., et al., Macrophages that have ingested apoptotic cells in vitro inhibit proinflammatory cytokine production through autocrine/paracrine mechanisms involving TGF-beta, PGE2, and PAF. J Clin Invest, 1998. 101(4): p. 890-8.
102. McDonald, P. P., et al., Transcriptional and translational regulation of inflammatory mediator production by endogenous TGF-beta in macrophages that have ingested apoptotic cells. J Immunol, 1999. 163(11): p. 6164-72.
103. Stuart, L. M., et al., Inhibitory effects of apoptotic cell ingestion upon endotoxin-driven myeloid dendritic cell maturation. J Immunol, 2002. 168(4): p. 1627-35.
104. Woo, J. J., et al., The complement system in schizophrenia: where are we now and what's next? Mol Psychiatry, 2020. 25(1): p. 114-130.
105. McGeer, P. L., M. Lee, and E. G. McGeer, A review of human diseases caused or exacerbated by aberrant complement activation. Neurobiol Aging, 2017. 52: p. 12-22.
106. Sim, R. B. and A. Laich, Serine proteases of the complement system. Biochem Soc Trans, 2000. 28(5): p. 545-50.
107. Cheng, L. L., et al., [Effect of C5-siRNA silencing receptor C5 on myocardial ischemia injury in rats]. Nan Fang Yi Ke Da Xue Xue Bao, 2010. 30(6): p. 1486-8.
108. Ebrahimi, K. B., et al., Decreased membrane complement regulators in the retinal pigmented epithelium contributes to age-related macular degeneration. J Pathol, 2013. 229(5): p. 729-42.
109. Bora, N. S., et al., Complement activation via alternative pathway is critical in the development of laser-induced choroidal neovascularization: role of factor B and factor H. J Immunol, 2006. 177(3): p. 1872-8.
110. Tang, K., et al., Protective effect of C5 shRNA on myocardial ischemia-reperfusion injury in rats. Can J Physiol Pharmacol, 2012. 90(10): p. 1394-402.
111. Hattori, Y., et al., Transdermal Delivery of Small Interfering RNA with Elastic Cationic Liposomes in Mice. J Pharm (Cairo), 2013. 2013: p. 149695.
112. Mehta, G., et al., A New Approach for the Treatment of Arthritis in Mice with a Novel Conjugate of an Anti-05aR1 Antibody and C5 Small Interfering RNA. J Immunol, 2015. 194(11): p. 5446-54.
113. Kusner, L. L., et al., Investigational RNAi Therapeutic Targeting C5 Is Efficacious in Pre-clinical Models of Myasthenia Gravis. Mol Ther Methods Clin Dev, 2019. 13: p. 484-492.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of increasing fibroblast therapeutic activity or the therapeutic activity of fibroblast derived products for, or in, an individual, comprising the steps of:
a. optionally assessing a potential for complement activation in an individual;
b. modulating complement activation in the individual; and
c. administering a therapeutically effective amount of fibroblasts and/or fibroblast derived products to the individual.

2. The method of claim 1, wherein said fibroblasts are derived from tissues selected from the group consisting of cord blood, Wharton's Jelly, placenta, bone marrow, adipose tissue, skin, deciduous teeth, nails, peripheral blood, omentum, and a combination thereof.

3. The method of claim 1 or claim 2, wherein said fibroblasts are allogeneic, autologous, or xenogeneic with respect to the individual.

4. The method of any one of claims 1-3, wherein said fibroblast-derived products comprise exosomes derived from fibroblasts.

5. The method of any one of claims 1-4, wherein said fibroblasts are plastic adherent.

6. The method of any one of claims 1-5, wherein said fibroblasts express markers selected from the group consisting of CD73, CD105, stage-specific embryonic antigens (SSEA) 3, SSEA 4, Tra-1-60, Tra-1-81, Oct-3/4, Cripto, gastrin-releasing peptide (GRP) receptor, podocalyxin-like protein (PODXL), Rex-1, GCTM-2, Nanog, telomerase reverse transcriptase (hTERT), CD9, CD13, CD29, CD44, CD166, DAZL, Runx-1, and a combination thereof.

7. The method of any one of claims 1-6, wherein said fibroblasts are reprogrammed to possess a more immature phenotype than fibroblasts that are not reprogrammed.

8. The method of claim 7, wherein the fibroblasts that are reprogrammed express one or more pluripotency markers.

9. The method of claim 8, wherein the pluripotency markers comprise a) OCT-4; b) NANOG; c) SOX-2; d) hTERT; e) acetylated histones; and f) a combination thereof.

10. The method of any one of claims 7-9, wherein said reprogramming comprises a nuclear transfer, a cytoplasmic transfer, contacting the fibroblasts with one or more DNA methyltransferase inhibitors, contacting the fibroblasts with one or more histone deacetylase inhibitors, contacting the fibroblasts with one or more GSK-3 inhibitors, dedifferentiation by alteration of extracellular conditions, or a combination thereof.

11. The method of claim 10, wherein said nuclear transfer comprises introducing nuclear material to substantially enucleated cell.

12. The method of claim 11, wherein the substantially enucleated cell is an oocyte.

13. The method of any one of claims 10-12, wherein said cytoplasmic transfer comprises introducing cytoplasmic contents from a cell with a dedifferentiated phenotype into a cell with a differentiated phenotype, wherein the cell with a differentiated phenotype substantially reverts to a dedifferentiated phenotype.

14. The method of claim 13, wherein the cells with a differentiated phenotype are fibroblasts.

15. The method of any one of claims 10-14, wherein said DNA demethylating agent comprises 5-azacytidine, psammaplin A, zebularine, or a combination thereof.

16. The method of any one of claims 10-15, wherein said histone deacetylase inhibitor comprises valproic acid, trichostatin-A, trapoxin A, depsipeptide, or a combination thereof.

17. The method of any one of claims 10-16, wherein the alteration of one or more extracellular conditions comprises culturing the fibroblasts in a pH from 5-7, culturing the fibroblasts in hypoxia, and/or culturing the fibroblasts in media conditioned by pluripotent stem cells.

18. The method of any one of claims 1-17, wherein said fibroblasts are selected from cells in a side population.

19. The method of claim 18, wherein said side population cells are identified based on expression of multidrug resistance transport protein (ABCG2) and/or an ability to efflux one or more intracellular dyes.

20. as the method of claim 19, wherein the dye is rhodamine-123 and/or Hoechst 33342.

21. The method of any one of claims 18-20, wherein the side population cells are derived from tissues selected from the group consisting of pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

22. The method of any one of claims 1-21, wherein said fibroblasts are obtained or enriched from blood.

23. The method of claim 22, wherein the fibroblasts obtained or enriched from blood are obtained or enriched from mobilized blood.

24. The method of claim 23, wherein the mobilized blood is derived from blood that has been administered one or more mobilizing agents and/or from an individual that has been administered one or more mobilizing agents.

25. The method of claim 24, wherein said mobilizing agent is selected from the group consisting of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, one or more small molecule antagonists of SDF-1, and a combination thereof.

26. The method of claim 24 or 25, wherein said individual is provided an effective amount of one or more mobilization therapies comprising a therapy selected from the group consisting of exercise, hyperbaric oxygen, autohemotherapy by ex vivo ozonation of peripheral blood, induction of SDF-1 secretion in an anatomical area outside of the bone marrow, and a combination thereof.

27. The method of any one of claims 1-26, wherein one or more antioxidants are also administered at a therapeutically sufficient concentration to the individual.

28. The method of claim 27, wherein said antioxidant is selected from the group consisting of ascorbic acid or one or more derivatives thereof, alpha tocopherol or one or more derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, superoxide dismutase, and a combination thereof.

29. The method of claim 27 or claim 28, wherein said antioxidant is administered prior to administration of fibroblasts and/or fibroblast-derived products at a concentration sufficient to reduce an inhibitory effect of oxidative stress on the fibroblast therapeutic activity.

30. The method of any one of claims 27-29, wherein said antioxidant is administered concurrently with the fibroblasts and/or fibroblast-derived products.

31. The method of any one of claims 27-29, wherein said antioxidant is administered subsequent to the fibroblasts and/or fibroblast-derived products cell administration.

32. The method of any one of claims 1-31, wherein modulating complement activation comprises administering to the individual at least one composition that inhibits the formation of terminal complement complex or C5a.

33. The method of claim 32, wherein at least one of the compositions that inhibits the formation of terminal complement complex or C5a comprises a whole antibody or an antibody fragment.

34. The method of claim 33, wherein the whole antibody or antibody fragment comprises a human, humanized, chimerized, or deimmunized antibody or antibody fragment.

35. The method of claim 33 or claim 34, wherein the whole antibody or antibody fragment inhibits cleavage of complement C5.

36. The method of any one of claims 33-35, wherein the antibody fragment is selected from the group consisting of an Fab, an F(ab′)₂, an Fv, a domain antibody, a single-chain antibody, and a combination thereof.

37. The method of any one of claims 33-36, wherein said antibody fragment is pexelizumab.

38. The method of any one of claims 33-35, wherein said whole antibody is eculizumab.

39. The method claim 38, wherein a therapeutically effective amount of eculizumab is administered to the individual once every 2 weeks.

40. The method of any one of claims 1-39, wherein modulating complement activation comprises administering a composition selected from the group consisting of i) soluble complement receptor, ii) CD59, iii) CD55, iv) CD46, v) an antibody to C5, C6, C7, C8, or C9, and vi) a combination thereof.

41. The method of any one of claims 1-40, wherein the fibroblast therapeutic activity comprises an ability of the fibroblasts and/or fibroblast-derived products to home to an area of tissue injury in the individual.

42. The method of claim 41, wherein said tissue injury comprises activation of the coagulation cascade.

43. The method of claim 41 or 42, wherein said tissue injury comprises loss of mitochondrial activity.

44. The method of claim 43, wherein the mitochondrial activity is measured as the ability to generate ATP.

45. The method of any one of claims 41-44, wherein said tissue injury comprises activation of tissue factor expression in cells of the tissue.

46. The method of any one of claims 41-45, wherein said tissue injury comprises induction of ischemia.

47. The method of any one of claims 41-46, wherein said tissue injury comprises reduction in ATP usage in damaged tissue cells.

48. The method of any one of claims 41-47, wherein said tissue injury comprises reduction in release of tissue adenosine.

49. The method of any one of claims 41-48, wherein the ability of the fibroblast and/or fibroblast-derived products to home is facilitated by an increased expression of at least one homing receptor in the fibroblasts.

50. The method of claim 49, wherein the homing receptor comprises CXCR-4.

51. The method of claim 49 or 50, wherein the homing receptor comprises CCR-5.

52. The method of claim 49, 50, or 51, wherein the homing receptor comprises VEGF receptor 2.

53. The method of any one of claims 1-52, wherein said fibroblast therapeutic activity comprises an ability to suppress a pathological immune response in the individual.

54. The method of claim 53, wherein said pathological immune response comprises tissue destruction, loss of function, and/or fibrosis.

55. The method of claim 53 or 54, wherein said pathological immune response is associated with production of interleukin-17.

56. The method of claim 55, wherein said production of interleukin-17 is mediated by an increased number of Th17 cells and/or increased activity of Th17 cells.

57. The method of any one of claims 53-56, wherein said pathological immune response comprises neutrophil activation in the individual.

58. The method of any one of claims 53-57, wherein said pathological immune response comprises a reduction in neutrophil apoptosis.

59. The method of any one of claims 53-58, wherein said pathological immune response comprises macrophage activation.

60. The method of claim 59, wherein said macrophage activation comprises an increase of nitric oxide and/or oxygen free radicals released by macrophages.

61. The method of claim 60, wherein said macrophages comprise M1 macrophages.

62. The method of any one of claims 59-61, wherein said macrophage activation comprises increased production of matrix metalloproteases.

63. The method of any one of claims 59-62, wherein said macrophage activation comprises exposure of immunogenic epitopes that activate natural antibodies.


SEQUENCE LISTING (2 sequences)

SEQ ID NO: 1 - RNA, 21 nt, Artificial Sequence
  Synthetic Polynucleotide
aagcaagauauuuuuauaaua

SEQ ID NO: 2 - RNA, 23 nt, Artificial Sequence
  Synthetic Polynucleotide
uauuauaaaaauaucuugcuuuu